(19) United States
(12) Patent Application Publication
(10) Pub. No.: US 2006/0143719 A1
(43) Pub. Date: Jun. 29, 2006

(54) Use of golden hamster as infectivity model of SARS

(76) Inventors: Philippe Marianneau, Lyon (FR); Vincent Deubel, Paris (FR); Hugues Contamin, Biuday (FR); Ingrid Marendat, Lyon (FR); Philippe Loth, Villeurbanne (FR); Marie Claude Georges-Courbot, Lyon (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/204,393

(22) Filed: Aug. 16, 2005

Related U.S. Application Data
(60) Provisional application No. 60/602,318, filed on Aug. 18, 2004.

(51) Int. Cl. A01K 67/027

(52) U.S. Cl. 800/9; 800/14

(57) ABSTRACT

A model system for sudden acute respiratory syndrome infection (SARS) in humans, comprising a non-human animal infected with a SARS-causing coronavirus (CoV), wherein the non-human animal is a golden hamster.

### CROSS-REFERENCE TO RELATED APPLICATIONS

This application is based on and claims the benefit of U.S. Provisional Application No. 60/602,318, filed Aug. 18, 2004, (Attorney Docket No. 3495.6100) The entire disclosure of this application is relied upon and incorporated by reference herein.

### FIELD OF THE INVENTION

This invention relates to the use of a rodent, namely a golden hamster or mice deficient in interferon alpha and interferon beta receptors as infectivity models of sudden acute respiratory syndrome (SARS) infection. This invention also relates to the use of these animal models to test the efficacy of antiviral drugs and vaccine candidates.

### BACKGROUND OF THE INVENTION

An outbreak of a novel infectious disease first emerged in Guangdong Province in south-east China in November 2002; from there, the severe acute respiratory syndrome (SARS) spread to various parts of the world in March 2003. An unprecedented international collaborative effort led by the World Health Organisation (WHO) resulted in the identification of a novel coronavirus (SARS-CoV) that was confirmed as the causative agent of SARS within only a few weeks¹.

While the last chain of human-to-human transmission was reported broken in July 2003, following the strict application of different infection control measures, there is uncertainty as to whether SARS will return. Genome sequence data proved that SARS-CoV is distinct from any previously known human or animal coronavirus. It probably originates from an hitherto unknown animal host, and for some unknown reason, developed the ability to infect humans. Studies conducted in wildlife and domestic animal markets in Guangdong demonstrated closely related coronaviruses in different animal species²; however the exact reservoir of this virus remains unknown.

So far, two animal models for SARS have been described, the cynomolgus macaque (Macaca fascicularis) and the ferret (Mustela furo) models^{3,4}. In both species, SARS-CoV causes pathogenicity. In addition, the domestic cat (Felix domesticus) is susceptible to infection, but does not develop illness⁴. Due to the difficulties of doing research in non-human primates, the availability of a small animal model easy to manipulate would be useful to initiate studies on potential anti-viral drugs and on vaccine candidates against SARS-CoV.

### SUMMARY OF THE INVENTION

Accordingly, this invention provides a model system for sudden acute respiratory syndrome infections (SARS) in humans, comprising a non-human animal infected with a SARS-causing coronavirus (CoV), wherein a non-human animal is a golden hamster. In one embodiment of the invention, the infected animal contains antibodies to the coronavirus. In another embodiment of the invention, the animal contains viral RNA of the coronavirus.

This invention provides a similar model system in which the non-human animal is a mouse deficient in interferon alpha and interferon beta receptors.

This invention also provides a method of preparing a host non-human animal as a model system for SARS infection in humans, wherein the method comprises administering to the animal as SARS-causing coronavirus in an amount sufficient to produce detectable antibodies to the coronavirus or to detect viral RNA coronavirus in the animal, wherein the animal is a golden hamster. In preferred embodiments of the invention, the animal is infected with the coronavirus by intraperitoneal or intranasal administration.

In an alternative embodiment of the invention, sera from the infected animal can be collected at several days post-infection to monitor viral RNA or antibodies against the coronavirus. Preferred dosages for infecting the animal are 2×107 pfu of the coronavirus when administered intraperitoneally, and 8×105 pfu of the coronavirus when administered intranasally.

In addition, this invention provides an antibody raised in a golden hamster, wherein the antibody specifically recognizes SARS-causing coronavirus. In a preferred embodiment of the invention, the antibody is a neutralizing polyclonal antibody. This invention also provides a method for producing polyclonal antibodies against the SARS-causing coronavirus. The method comprises infecting a golden hamster with the coronavirus by intranasal or intraperitoneal administration, and collecting sera containing the polyclonal antibodies.

Further, this invention provides a method for screening an antiviral drug or vaccine product, wherein the method comprises administering the antiviral drug or vaccine product to a golden hamster at the same time as infecting the animal with SARS coronavirus, collecting sera of the animal at several days post-infection to monitor viral RNA and/or antibodies against SARS coronavirus, comparing the quantified viral RNA and/or antibodies with the quantified ones of an untreated infected animal, and selecting the antiviral drug or vaccine product that induces a decrease of the quantity of viral RNA and/or a reduced neutralising antibodies titres.

### DETAILED DESCRIPTION OF THE INVENTION

Golden hamsters and inbred mice were infected with the coronavirus responsible of the severe acute respiratory syndrome (CoV-SARS). Viral RNA were detected in sera and lungs from animals and persisted in the presence of neutralizing anti-CoV-SARS antibodies. Mice showed a lower susceptibility to the virus, but hamsters are a useful model in initial studies to test the efficacy of antiviral drugs or vaccine candidates against SARS.

The non-human animal used as a host in this invention is a golden hamster. Adult animals of about 3 weeks to about 3 months of age have been found to be suitable. Animals of other ages can be employed, it being understood, however, that suckling or infant animals would not be suitable for vaccine or drug trials. There are no known limitations on the strain used or the phenotype of the animal. Thus, it will be understood that other hamster species can be employed.

The animal should be healthy and preferably free of other viral, bacterial, or other infections. The animal may or may not be immunosuppressed, such as by administration of an immunosuppressive agent or an immunosuppressive treatment.

A viral inoculum for infecting the animal model of SARS infection can be prepared according to standard methods known in the art. One appropriate procedure is described hereinafter.

Infection of the animal model can be accomplished by any route, including, but not limited to, intravenous, intraperitoneal, and subcutaneous routes. Preferred routes of administration are intranasal (IN) and intraperitoneal (IP).

The dosage of the SARS pathogen administered to the animal can be varied. Typically, the animal will receive a dose that is within a range of about 104 orders or magnitude below to about 104 orders of magnitude above the ID (infectious dose) 50 of the pathogen. Dosages can thus be determined with a minimum of experimentation. Examples of suitable dosages are provided hereinafter.

In one embodiment of this invention, the infectivity and pathogenicity of SARS-CoV was investigated in different laboratory animals: eight-week-old male golden hamsters (Janvier Company, St Genest, St Isles, France), inbread 129Sv mice, and inbread IFNAR-1−/− deficient 129Sv mice (Mus musculus) (Pasteur Institute, Paris), the latter lacking a functional interferon alpha/beta receptor and highly susceptible to many different viruses5.

SARS-CoV strain isolated from the Frankfurt index case6 was used. Virus stock was prepared by harvesting the cell culture supernatant from Vero E6 cells five days post-infection (p.i.) with a multiplicity of infection of 0.01 plaque forming unit (pfu)/cell and by collecting the cell supernatant five days post-infection. Its virus titre was 4×107 pfu/ml determined by plaque assay stained with crystal violet.

Animals were inoculated and sera were collected after gaseous anesthesia in an induction chamber using isofurane. Four male golden hamsters were inoculated with 2×107 pfu of virus by the intraperitoneal (IP) route and four with 8×105 pfu by the intranasal (IN) route. Two non-infected hamsters served as control. Four IFNAR-1−/− deficient mice and four 129Sv mice were inoculated IP with 8×106 pfu, and four IFNAR-1−/− mice IN with 8×105 pfu. Three non-infected mice served as control.

Body temperatures were checked daily using implanted programmable temperature transponder IPTT-200 and an IPTT Das 5007 pocket scanner (PLEXX, The Netherlands). Hamsters' sera were collected at several days p.i. to monitor the viral RNA and SARS-CoV antibodies. One mouse of each group was euthanasied at different days p.i. and blood and lung tissues were collected for detection of viral RNA and anti-SARS-CoV antibodies.

Virus titration was attempted on all mice and hamster sera collected as well as on lungs from two IP inoculated hamsters euthanasied at day 37 p.i., and IP or IN inoculated mice, on Vero cells starting at 1/10 dilution. Plates were read 5 days post-infection after crystal violet staining. Virus isolation was also attempted on the undiluted sera of IP and IN inoculated hamsters.

RT-PCR was performed on serum and organ samples of infected and non-infected hamsters after RNA extraction using QIAamp viral RNA mini kit (Qiagen). Single-round and nested-PCR were performed on sera and lungs using the previously described BNIoutS2/BNIoutAS and BNIinS/BNIinAS primers localised in the L gene6.

Anti-SARS-CoV IgG antibodies were tested by 96-well microplate Elisa coated with crude lysate of SARS-CoV-infected Vero cells harvested 5 days after infection and of non-infected cells as controls.

Neutralising antibodies were determined by incubating serial two-fold dilutions of serum with 50 pfu of CoV for one hour at 37° C. and adding the mixture to Vero cells in 96-wells plates. On day 5, the plates were read after crystal violet coloration, and the neutralising antibody titre determined as the last dilution of the serum that inhibited the destruction of the cell layer by the virus.

The mouse models tested in this study showed a lower susceptibility to SARS-CoV than did golden hamsters. Moreover, the low susceptibility of IFNAR-1−/− mice did not differ from that of 129Sv mice, suggesting that a pathway different of that of type I interferon may restrict virus replication in these animals.

More particularly, none of the inoculated animals developed signs of disease. However, all inoculated hamsters and mice developed anti-SARS-CoV-specific antibodies by ELISA as well as neutralising antibody with titres ranging from 160 to ≧640 in hamsters and 20 to 160 for mice, independent of the route of inoculation (Tables 1 and 2). None of the control animals had detectable anti-SARS-CoV antibodies (data not shown).

TABLE 1
Serological and RT-PCR results from hamsters inoculated with
SARS-CoV.

| | Nb of | | | | RT-PCR |
|---|---|---|---|---|---|
| I R¹ | animals² | Days PI³ | OD IgG⁴ | NT Ab titre⁵ | in sera⁶ |
| IP | 2 | 3 | 0.01 | nd⁷ | Pos |
| IP | 2 | 6 | 1.94 ± 0.23 | nd | Pos |
| IP | 4 | 11 | 2.14 ± 0.15 | 320 | Pos |
| IP | 4 | 23 | 2.19 ± 0.10 | 640 | Pos |
| IP | 2⁸ | 37 | 2.03 ± 0.03 | ≧640 | Pos |
| IP | 2 | 47 | 1.99 ± 0.04 | ≧640 | Pos |
| IN | 2 | 3 | 0.45 ± 0.02 | nd | Pos |
| IN | 2 | 6 | 1.3 ± 0.11 | 160 | Pos |
| IN | 4 | 11 | 1.37 ± 0.08 | 320 | Neg |
| IN | 4⁸ | 23 | 1.69 ± 0.05 | 640 | Neg |

¹Inoculation route,
²Number of animal tested,
³Number of days post inoculation,
⁴Anti SARS-CoV IgG detected by Elisa test against crude antigens prepared on SARS-CoV-infected Vero cells (mean ± standard deviation of optical density obtained in sera diluted 1:100),
⁵Titre of neutralising antibodies (the neutralising antibody titre was determined as the last dilution of the serum that inhibited the destruction of the Vero cell layer by the CoV-SARS),
⁶Results of RT-PCR (Single-round and nested-PCR were performed on sera and lungs as previously described⁵),
⁷not done,
⁸Animals were euthanasied.

TABLE 2
Serological and RT-PCR results from mice inoculated with SARS-CoV.

| | | Days | | NT Ab | RT-PCR | RT-PCR |
|---|---|---|---|---|---|---|
| Animal No¹ | I R¹ | PI³ | OD IgG⁴ | titre⁵ | in sera⁶ | in lungs⁶ |
| IFNAR 1 | IP | 6 | 0.01 | nd⁷ | Neg | Pos |
| IFNAR 2 | IP | 11 | 1.02 | 80 | Neg | Neg |
| IFNAR 3 | IP | 20 | 1.55 | nd | Neg | nd |
| IFNAR 4 | IP | 20 | 1.11 | 40 | Neg | Pos |
| IFNAR 1 | IN | 6 | 0.45 | 80 | Pos | Neg |
| IFNAR 2 | IN | 11 | 1.3 | 80 | Neg | Pos |
| IFNAR 3 | IN | 23 | 1.37 | 80 | Neg | nd |
| IFNAR 4 | IN | 23 | 1.69 | 80 | Neg | Pos |
| 129Sv 1 | IN | 6 | 0.01 | 20 | Neg | nd |
| 129Sv 2 | IN | 11 | 0.91 | 80 | Neg | Neg |
| 129Sv 3 | IN | 19 | 0.93 | 160 | Neg | Pos |
| 129Sv 4 | IN | 19 | 1.39 | 160 | Neg | nd |

¹Species and number of animal,
²Inoculation route,
³Day post inoculation and of mouse euthanasia,
⁴Anti SARS-CoV IgG detected by Elisa test (mean ± standard deviation of optical density obtained in sera diluted 1:100),
⁵Titre of neutralising antibodies,
⁶Results of RT-PCR,
⁷not done.

No virus could be isolated from sera or organs in Vero cell cultures (data not shown), but viral RNA was detected by RT-PCR. All sera from IP inoculated hamsters remained positive from 3-6 days to 47 days p.i. by RT-PCR. However, only early samples were found positive in IN inoculated hamsters (Table 1). Only one early sample was found positive by RT-PCR in one IFNAR-1−/− mouse (Table 2). The absence of detectable viral RNA in mouse sera—with one exception—might explain their lower neutralising antibodies as compared to hamsters.

Both lung samples collected at day 37 p.i. from hamsters inoculated IP were positive by RT-PCR (data not shown), as well as five out of eight lungs of IFNAR-1−/− and 129Sv mice collected between 6 and 23 days (Table 2). These results suggest that the lungs are an important site of virus replication in both types of animals. In addition, the faeces of two hamsters inoculated IP were positive by RT-PCR until day 37 p.i. (data not shown).

The following table presents additional results in hamsters.

| | | HAMSTERS | | | | |
|---|---|---|---|---|---|---|
| route of inoculation | day at autopsy | Animal | Techniques | serum | Feces | Urine |
| | D11 | H1 = Mock | PCR | − | − | − |
| | | | Nested PCR | − | | − |
| | | | ELISA IgG | 0.001 | x | x |
| | D1 | H3 | PCR | + | + | − |
| | | | Nested PCR | + | | + |
| | | | ELISA IgG | 0.003 | x | x |
| | D1 | H4 | PCR | + | + | − |
| | | | Nested PCR | + | | + |
| | | | ELISA IgG | −0.003 | x | x |
| | D2 | H5 | PCR | + | + | − |
| | | | Nested PCR | + | | + |
| | | | ELISA IgG | −0.007 | x | x |
| | D2 | H6 | PCR | + | + | − |
| | | | Nested PCR | + | | − |
| | | | ELISA IgG | −0.007 | x | x |
| | D3 | H7 | PCR | + | + | − |
| | | | Nested PCR | + | | + |
| | | | ELISA IgG | −0.003 | x | x |
| | D3 | H8 | PCR | + | + | − |
| | | | Nested PCR | + | | + |
| | | | ELISA IgG | 0.025 | x | x |
| | D4 | H9 | PCR | + | + | + |
| | | | Nested PCR | + | | + |
| | | | ELISA IgG | 0.554 | x | x |
| | D4 | H10 | PCR | | + | − |
| | | | Nested PCR | + | | + |
| | | | ELISA IgG | 0.591 | x | x |
| | D5 | H11 | PCR | + | + | + |
| | | | Nested PCR | + | | + |
| | | | ELISA IgG | 1.178 | x | x |
| | D5 | H12 | PCR | + | + | + |
| | | | Nested PCR | + | | + |
| | | | ELISA IgG | 1.318 | x | x |
| | D6 | H13 | PCR | + | + | + |
| | | | Nested PCR | + | | + |
| | | | ELISA IgG | 1.617 | x | x |
| | D6 | H14 | PCR | − | − | + |
| | | | Nested PCR | + | | + |
| | | | ELISA IgG | 1.745 | x | x |
| | D7 | H15 | PCR | + | + | − |
| | | | Nested PCR | + | | − |
| | | | ELISA IgG | 1.998 | x | x |
| | D7 | H16 | PCR | + | + | − |
| | | | Nested PCR | + | | + |
| | | | ELISA IgG | 1.88 | x | x |
| | D8 | H17 | PCR | + | + | + |
| | | | Nested PCR | + | | + |
| | | | ELISA IgG | 1.949 | x | x |
| | D8 | H18 | PCR | − | − | − |
| | | | Nested PCR | + | | + |
| | | | ELISA IgG | 1.873 | x | x |
| | D9 | H19 | PCR | − | + | − |
| | | | Nested PCR | + | | + |
| | | | ELISA IgG | 1.927 | x | x |
| | D9 | H20 | PCR | − | + | + |
| | | | Nested PCR | + | | + |
| | | | ELISA IgG | 1.921 | x | x |
| | D16 | H21 | PCR | − | + | − |
| | | | Nested PCR | + | | + |
| | | | ELISA IgG | 2.015 | x | x |
| | D16 | H22 | PCR | + | + | − |
| | | | Nested PCR | + | | + |
| | | | ELISA IgG | 2.025 | x | x |
| | D11 | H28 = Mock | PCR | − | − | − |
| | | | Nested PCR | − | | − |
| | | | ELISA IgG | −0.002 | x | x |
| | D11 | H29 = Mock | PCR | − | − | − |
| | | | Nested PCR | − | | − |
| | | | ELISA IgG | 0.001 | x | x |
| | D1 | H30 | PCR | + | + | − |
| | | | Nested PCR | + | | − |
| | | | ELISA IgG | −0.001 | x | x |
| | D1 | H31 | PCR | + | + | − |
| | | | Nested PCR | + | | − |
| | | | ELISA IgG | −0.004 | x | x |
| | D2 | H32 | PCR | + | + | − |
| | | | Nested PCR | + | | − |
| | | | ELISA IgG | 0 | x | x |
| | D2 | H33 | PCR | + | + | − |
| | | | Nested PCR | + | | + |
| | | | ELISA IgG | 0 | x | x |
| | D3 | H34 | PCR | | + | − |
| | | | Nested PCR | + | | − |
| | | | ELISA IgG | 0 | x | x |
| | D3 | H35 | PCR | | + | − |
| | | | Nested PCR | + | | − |
| | | | ELISA IgG | −0.001 | x | x |
| | D4 | H36 | PCR | | + | − |
| | | | Nested PCR | + | | − |
| | | | ELISA IgG | 0.219 | x | x |
| | D4 | H37 | PCR | + | + | − |
| | | | Nested PCR | + | | − |
| | | | ELISA IgG | 0.136 | x | x |
| | D5 | H38 | PCR | + | + | − |
| | | | Nested PCR | + | | − |
| | | | ELISA IgG | 1.036 | x | x |
| | D5 | H39 | PCR | + | + | − |
| | | | Nested PCR | + | | + |
| | | | ELISA IgG | 0.183 | x | x |
| | D6 | H40 | PCR | + | + | − |
| | | | Nested PCR | + | | − |
| | | | ELISA IgG | 1.637 | x | x |
| | D6 | H41 | PCR | + | + | (+) |
| | | | Nested PCR | + | | + |
| | | | ELISA IgG | 1.602 | x | x |
| | D7 | H42 | PCR | − | − | − |
| | | | Nested PCR | + | | − |
| | | | ELISA IgG | 1.662 | x | x |
| | D7 | H43 | PCR | − | − | − |
| | | | Nested PCR | + | | − |
| | | | ELISA IgG | 1.725 | x | x |
| | D8 | H44 | PCR | − | − | − |
| | | | Nested PCR | + | | − |
| | | | ELISA IgG | 1.84 | x | x |
| | D8 | H45 | PCR | (+) | (+) | − |
| | | | Nested PCR | + | | − |
| | | | ELISA IgG | 1.802 | x | x |
| | D9 | H46 | PCR | − | − | − |
| | | | Nested PCR | − | | − |
| | | | ELISA IgG | 1.728 | x | x |
| | D9 | H47 | PCR | − | − | − |
| | | | Nested PCR | + | | − |
| | | | ELISA IgG | 1.59 | x | x |
| | D16 | H48 | PCR | − | − | − |
| | | | Nested PCR | + | | + |
| | | | ELISA IgG | 1.688 | x | x |
| | D16 | H49 | PCR | − | − | − |
| | | | Nested PCR | − | | − |
| | | | ELISA IgG | 1.677 | x | x |

These additional results were obtained by the same protocol and show a daily study of the virus present up to 9 days pi and a complete study on the urine of infected hamsters. These results show that the virus persists for a longer time in feces and urine in hamsters infected by IP route than in hamsters infected by IN route. Hamsters infected by the IP route are then a preferred model as compared to hamsters infected by the IN route, in order to study the effect of potential antiviral drugs.

One ebodiment of a screening test for antiviral drugs comprises injecting the drug to mbe tested at the same time as the virus into the animal. If the drug is active, it can be tested as a prophylactic drug (preventive treatment) and as a curative drug (administration of the drug at different times after infection to determine the period of time necessary to modify the biremia). In the hamster, the incubation period of SARS coronavirus is very short; the virus is detectable from twenty-four hours after the infection. Then, to reduce the viral load, the time to operate after outbreak of symptoms is very short. Nevertheless, when infection is made intraperitoneally in the hamster mode, the virus persists for more than three weeks. Thus, this model can be used to check whether antiviral drugs can eliminate virus from the animal earlier than three weeks.

In summary, it has been discovered that SARS-CoV infection can persist in golden hamsters and in mice, even in the presence of neutralising antibodies, a feature observed in presumed animal reservoirs of several viruses like hantaviruses, arenaviruses, or henipaviruses. However, no virus has been recovered from the samples, suggesting a low replication rate, or the presence of interfering particles, or of immune-complexed viruses. Viruses may appear to higher titers earlier than 6 days post-infection and then persist to low titers (Ref. 7). No symptoms were observed in any of the two rodent models tested. This result differs from the previous studies carried out on primates and ferrets, which had detectable virus in their sera and were susceptible to SARS-CoV infection^{3,4}.

Even though no pathology was observed, the golden hamsters infected IP is a relevant model for SARS-CoV infection and can be used in initial studies to test the efficacy of antiviral drugs or vaccine products for treating or preventing SARS infections. The efficiency of compounds can be assessed by a relative decrease or absence of viraemia detected by RT-PCR, absence of viral material in faeces, or reduced neutralising antibodies titres in comparison to untreated animals. However, comparative quantification of viral RNA in the samples of treated and non-treated animals is relevant in such studies.

### REFERENCES

The following references are incorporated by reference, in their entirety, herein.
1. Kuiken T, Fouchier R A, Schutten M, Rimmelzwaan G F, van Amerongen G, van Riel D, Laman J D, de Jong T, van Doornum G, Lim W, Ling A E, Chan P K, Tam J S, Zambon M C, Gopal R, Drosten C, van der Werf S, Escriou N, Manuguerra J C, Stohr K, Peiris J S, Osterhaus A D. Newly discovered coronavirus as the primary cause of severe acute respiratory syndrome. Lancet 2003;362 :263-70.
2. Guan Y, Zheng B J, He Y Q, Liu X L, Zhuang Z X, Cheung C L, Luo S W, Li P H, Zhang L J, Guan Y J, Butt K M, Wong K L, Chan K W, Lim W, Shortridge K F, Yuen K Y, Peiris J S, Poon L L. Isolation and Characterization of Viruses Related to the SARS Coronavirus from Animals in Southern China. Science. Published online Sep. 4, 2003. Abstract 1087139 (Science Express Report).
3. Fouchier R A, Kuiken T, Schutten M, van Amerongen G, van Doornum G J, van den Hoogen B G, Peiris M, Lim W, Stohr K, Osterhaus A D. Aetiology: Koch's postulates fulfilled for SARS virus. Nature 2003; 423:240.).
4. Martina B E, Haagmans B L, Kuiken T, Fouchier R A, Rimmelzwaan G F, Van Amerongen G, Peiris J S, Lim W, Osterhaus A D. SARS virus infection of cats and ferrets. Nature 2003;425:915.
5. Fiette L, Aubert C, Muller U, Huang S, Aguet M, Brahic M, Bureau J F. Theiler's virus infection of 129Sv mice that lack the interferon alpha/beta or interferon gamma receptors. J Exp Med. 1995, 181:2069-76.
6. Drosten C, Gunther S, Preiser W, van der Werf S, Brodt H R, Becker S, Rabenau H, Panning M, Kolesnikova L, Fouchier R A, Berger A, Burguiere A M, Cinatl J, Eickmann M, Escriou N, Grywna K, Kramme S, Manuguerra J C, Muller S, Rickerts V, Sturmer M, Vieth S, Klenk H D, Osterhaus A D, Schmitz H, Doerr H W. Identification of a novel coronavirus in patients with severe acute respiratory syndrome. N Engl J Med, 2003, 348:1967-76.
7. Subbarao, K, McAuliffe, J, Vogel L, Fable G, Fischer S, Tatti K, Packard M. Shieh W J, Zaki S, Murphy B. Prior infection and passive transfer of neutralizing antibody prevent replication of severe acute respiratory syndrome coronavirus in the respiratory tract of mice. J. Virol. 2004 1978: 3572-7.

What is claimed is:

1. A model system for sudden acute respiratory syndrome infection (SARS) in humans, comprising a non-human animal infected with a SARS-causing coronavirus (CoV), wherein the non-human animal is a golden hamster.

2. The model system of claim 1, wherein the animal contains antibodies to the coronavirus.

3. The model system as claimed in claim 1, wherein the animal contains viral RNA of the coronavirus.

4. A model system for sudden acute respiratory syndrome infection (SARS) in humans, comprising a non-human animal infected with a SARS-causing coronavirus (CoV), wherein the non-human animal is a mouse deficient in interferon alpha and interferon beta receptors.

5. The model system of claim 4, wherein the animal contains antibodies to the coronavirus.

6. The model system as claimed in claim 4, wherein the animal contains viral RNA of the coronavirus.

7. A method of preparing a host non-human animal as a model system for SARS infection in humans, wherein the method comprises administering to the animal as SARS-causing coronavirus in an amount sufficient to produce detectable antibodies to the coronavirus or to detect viral RNA coronavirus in the animal, wherein the animal is a golden hamster.

8. The method as claimed in claim 7, which comprises infecting the animal with the coronavirus by intraperitoneal or intranasal route and collecting sera of the animal at several days post-infection to monitor viral RNA or antibodies against the coronavirus.

9. The method as claimed in claim 7, which comprises infecting the animal with II×10⁷ pfu of the coronavirus intraperitoneally.

10. The method as claimed in claim 7, which comprises infecting the animal with 8×10⁵ pfu of the coronavirus intranasally.

11. An antibody that specifically recognizes SARS-causing coronavirus, wherein the antibody has been raised in a golden hamster.

12. The antibody as claimed in claim 11, which is a neutralizing polyclonal antibody.

13. A method for producing polyclonal antibodies against a SARS-causing coronavirus, wherein the method comprises infecting a golden hamster with the coronavirus by intranasal or intraperitoneal administration, and collecting sera containing the polyclonal antibodies.

14. A method for screening an antiviral drug or vaccine product, wherein the method comprises administering the antiviral drug or vaccine product to a golden hamster at the same time as infecting the said animal with SARS coronavirus, collecting sera of the animal at several days post-infection to monitor viral RNA and/or antibodies against SARS coronavirus, comparing the quantified viral RNA and/or antibodies with the quantified ones of an untreated infected animal, and selecting the antiviral drug or vaccine product that induces a decrease of the quantity of viral RNA and/or a reduced neutralising antibodies titres.
